(19) United States
(12) Patent Application Publication
(10) Pub. No.: US 2014/0039382 A1
(43) Pub. Date: Feb. 6, 2014

(54) Analyte Meter Module for Medication Delivery Device

(71) Applicants: Jonathan Fern, Alameda, CA (US); Mark P. Jesser, Austin, TX (US); Joel Goldsmith, Oakland, CA (US); Ronaldo F. Carreon, San Francisco, CA (US); Christopher A. Wilson, Fremont, CA (US); William J. Evans, San Francisco, CA (US); Matthew P. Presta, San Mateo, CA (US)

(72) Inventors: Jonathan Fern, Alameda, CA (US); Mark P. Jesser, Austin, TX (US); Joel Goldsmith, Oakland, CA (US); Ronaldo F. Carreon, San Francisco, CA (US); Christopher A. Wilson, Fremont, CA (US); William J. Evans, San Francisco, CA (US); Matthew P. Presta, San Mateo, CA (US)

(73) Assignee: Abbott Diabetes Care Inc., Alameda, CA (US)

(21) Appl. No.: 13/985,948

(22) PCT Filed: Dec. 21, 2011
(86) PCT No.: PCT/US11/66395

§ 371 Date: Sep. 19, 2013

Related U.S. Application Data
(60) Provisional application No. 61/444,055, filed on Feb. 17, 2011.

(51) Int. Cl. A61M 5/315; H05K 5/00

(52) U.S. Cl. CPC A61M 5/31546; H05K 5/0021
USPC 604/65; 235/462.11; 235/462.41; 361/729

(57) ABSTRACT

Presented herein is a modular analyte measurement system. The analyte measurement system includes an analyte meter and at least one modular attachment. In one embodiment, a modular attachment is provided to communicate with, and transmit/receive data from, one or more medication delivery device. Embodiments of the present invention relate to modular components of the analyte measurement system.

### CROSS-REFERENCE TO RELATED APPLICATIONS

Pursuant to 35 U.S.C. §119(e), this application claims priority to U.S. Provisional Patent Application No. 61/444,055 filed on Feb. 17, 2011, the disclosure of which is herein incorporated by reference in its entirety.

This application is also related to U.S. Provisional Patent Application No. 61/325,155, filed on Apr. 16, 2010; and U.S. Provisional Patent Application No. 61/444,058 filed on Feb. 17, 2011; the disclosures of which are incorporated herein by reference in their entirety.

### BACKGROUND OF THE INVENTION

1. The Field of the Invention

The present invention relates to analyte measurement systems. More specifically, the present invention relates to an analyte measurement system having an analyte meter with a modular attachment that communicates with a drug delivery system.

2. Background

One tool used in diabetes management is an analyte meter. An analyte meter is typically used to measure the blood glucose level of a user based on a sample of blood. The process of using an analyte meter is not complicated, and is often performed several times a day. First, the user inserts an analyte test strip into a test strip port of the meter. The user then lances her finger to obtain a small sample of blood. The blood sample is then placed onto the analyte test strip, and the meter analyzes the blood sample. The meter then typically displays a blood glucose level from the analysis.

Medication delivery devices, such as, for example, insulin injection pens (or syringes) are also available for diabetes management. Such devices are provided in order to allow the user to “self-manage” their diabetes treatment by injecting appropriate amounts of insulin throughout the day.

What is needed is an analyte meter that can communicate with, and transmit/receive data from, one or more medication delivery devices, such as an injection pen.

### BRIEF SUMMARY

Presented herein is a modular analyte measurement system. The analyte measurement system includes an analyte meter and at least one modular attachment. In one embodiment, a modular attachment is provided to communicate with, and transmit/receive data from, one or more medication delivery device. Embodiments of the present invention relate to modular components of the analyte measurement system.

### BRIEF DESCRIPTION OF THE FIGURES

The accompanying drawings, which are incorporated herein, form part of the specification. Together with this written description, the drawings further serve to explain the principles of, and to enable a person skilled in the relevant art(s), to make and use the present invention.

FIG. 1A provides a front-side view of an analyte measurement system in accordance with one embodiment presented herein.

FIG. 1B shows a back-side view of the analyte measurement system of FIG. 1A.

FIG. 2 illustrates a perspective view of a medication delivery device in accordance with one embodiment presented herein.

FIG. 3 illustrates one embodiment presented herein.

FIG. 4 illustrates a user practicing one embodiment presented herein.

FIG. 5 illustrates the medication delivery device of FIG. 2 in direct engagement with the analyte measurement system of FIGS. 1A and 1B.

FIG. 6A provides a front-side view of an analyte measurement system in accordance with another embodiment presented herein.

FIG. 6B illustrates a back-side view of the analyte measurement system of FIG. 6A.

FIG. 7 illustrates a perspective view of a medication delivery device in accordance with another embodiment presented herein.

FIG. 8 illustrates one embodiment presented herein.

FIG. 9 illustrates the medication delivery device of FIG. 7 in direct engagement with the analyte measurement system of FIGS. 6A and 6B.

FIG. 10 illustrates a user practicing one embodiment presented herein.

### DETAILED DESCRIPTION OF THE INVENTION

Before the embodiments of the present disclosure are described, it is to be understood that this invention is not limited to particular embodiments described, as such may, of course, vary. It is also to be understood that the terminology used herein is for the purpose of describing particular embodiments only, and is not intended to be limiting, since the scope of the embodiments of the invention will be limited only by the appended claims.

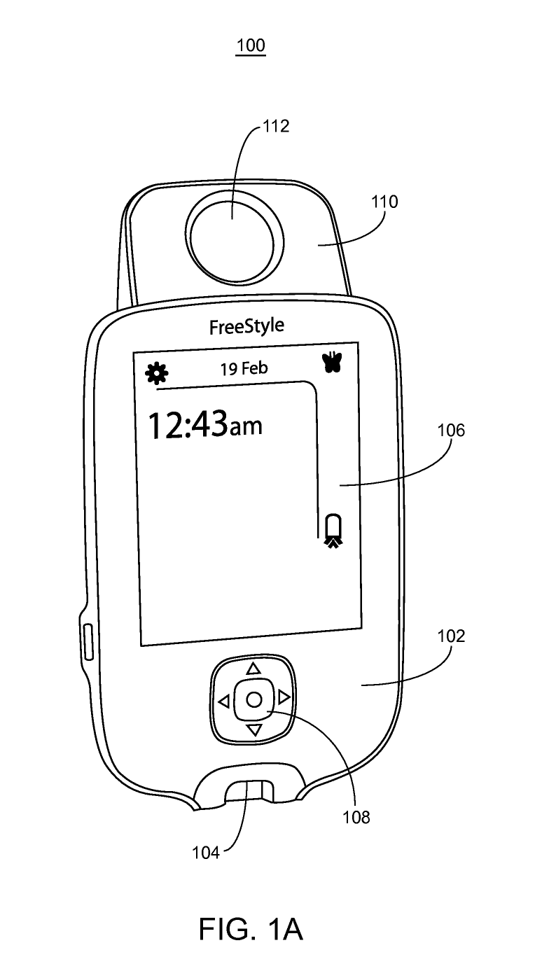

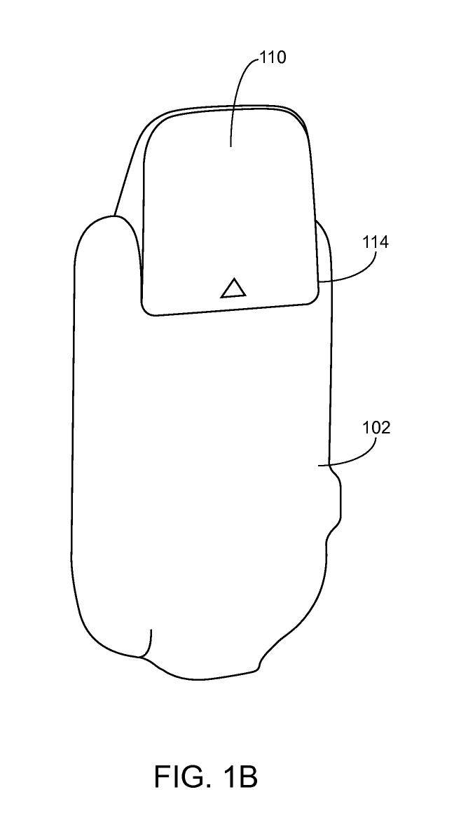

FIG. 1A provides a front-side view of an analyte measurement system 100 in accordance with one embodiment presented herein. FIG. 1B shows a back-side view of analyte measurement system 100. In one embodiment, analyte measurement system 100 includes an analyte meter 102. In turn, analyte meter 102 includes a test strip port 104, a display unit 106, and at least one control button 108. In practice, an analyte test strip (or sensor) is inserted into test strip port 104 in order to conduct an analyte test; for example, a blood glucose reading or a blood ketone reading. Meter 102 includes software to analyze the sample placed on the test strip, and the results of the analysis are typically displayed to the user via display unit 106. The user may also use control button 108 to provide appropriate instructions to meter 102.

In the embodiment shown, analyte measurement system 100 further includes a modular attachment 110. Modular attachment 110 is set within, and preferably interlocked within, a receptacle 114 in the housing of analyte meter 102. In one embodiment, for example, a snap-fit engagement may be provided between modular attachment 110 and receptacle 114. Electrical connections (not shown) are also provided between modular attachment 110 and meter 102 in order to transmit data and/or instructions between meter 102 and modular attachment 110.

Modular attachment 110 includes an external data transmission port 112. As will be further discussed below data transmission port 112 is used to communicate with an external device; such as, for example, a medication (drug) deliver device. In one embodiment, data transmission port 112 is an optical reader, such as an infrared optical reader. In other embodiments, data transmission port 112 may provide both a reader and a transmitter in order to both receive and send data or instructions. In another embodiment, data transmission port 112 includes a one-dimensional or two-dimensional bar code reader. The reader provided in data transmission port 112 may be configured to read human-readable or machine-readable output displays.

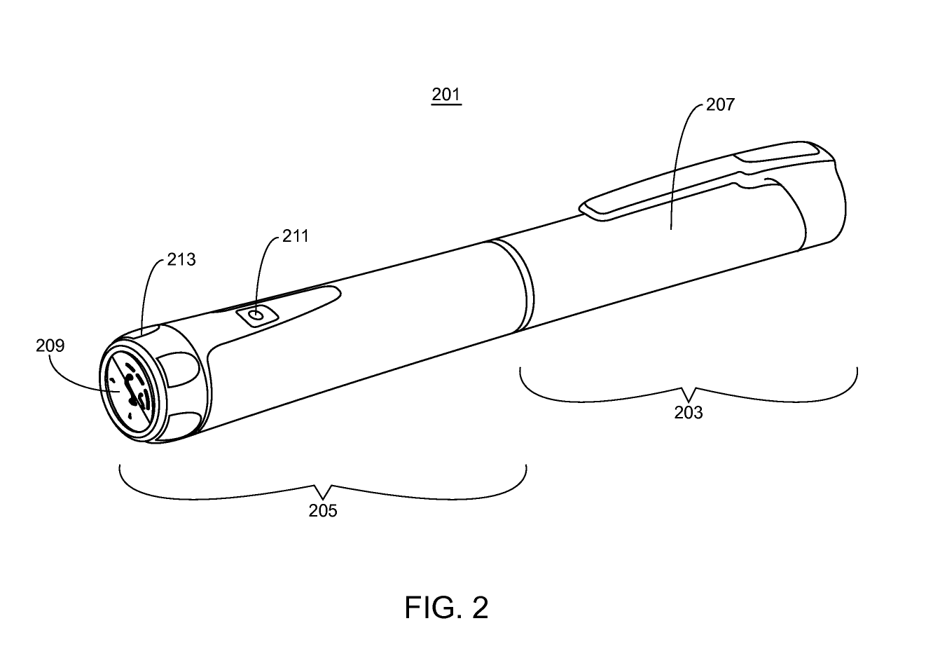

FIG. 2 illustrates a perspective view of a medication delivery device 201, in accordance with one embodiment presented herein. Medication delivery device 201 may be similar to commercially available insulin injection pens, and generally includes an injection end 203 and a data display/transmission end 205. Injection end 203 is shown with a removable cap 207. Underneath cap 207, the injection end 203 of medication delivery device 201 provides an injection needle or syringe. The data display/transmission end 205 of medication delivery device 201 includes a digital display screen 209, a dosage display screen 211, and a dosage control knob 213. Exemplary pens are described in, for example, U.S. Pat. Nos. 6,004,297; 6,235,004; 6,582,404; 7,195,616; 7,291,132; and 7,678,084; the disclosures of which are herein incorporated by reference in their entirety.

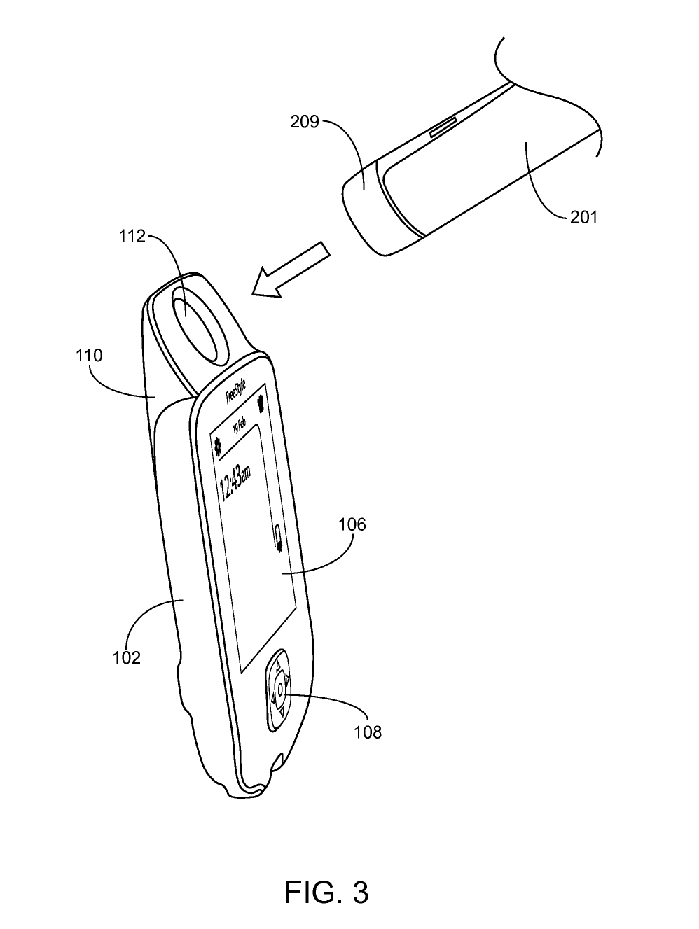

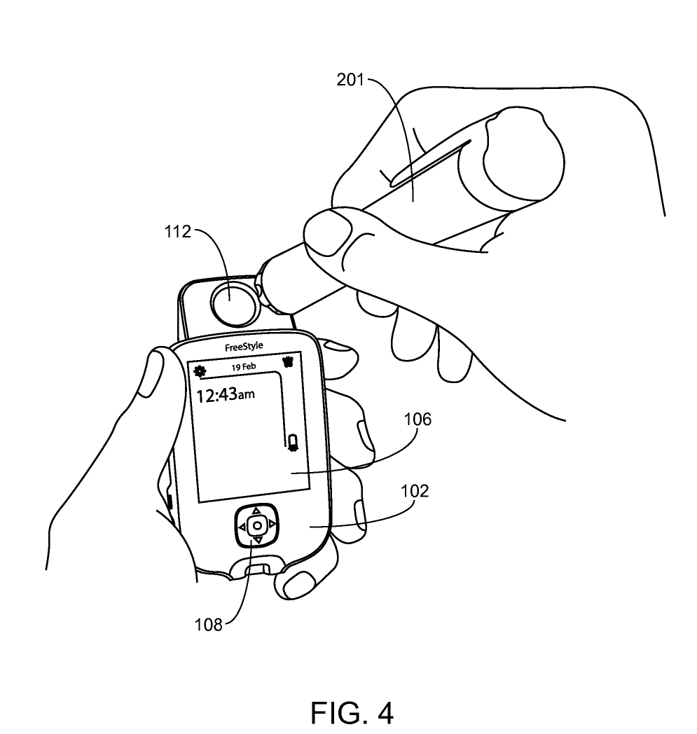

FIG. 3 illustrates one embodiment presented herein. FIG. 4 illustrates a user practicing the embodiment shown in FIG. 3. As shown in FIGS. 3 and 4, display screen 209 of medication delivery device 201 is presented to data transmission port 112 of analyte meter 102. Data and/or instructions may then be transmitted between medication delivery device 201 and analyte meter 102. The transmission may be provided wirelessly; by for example, WiFi, Bluetooth, IR, or equivalent wireless transmission means.

In one embodiment, dosage information is displayed by medication delivery device 201 via digital display screen 209. An optical reader provided in data transmission port 112 then reads the dosage information and transmits the dosage information to analyte meter 102. Analyte meter 102 may then include software to analyze, store, transmit, and/or display the received dosage information to the user. Analyte meter 102 may further include software to determine whether there is a need to change the dosage instructions, and then provide new dosage instructions to medication delivery device 201 via data transmission port 112. In another embodiment, analyte meter 102 includes software to provide the user with dosage instructions. The user can then modify their dosages (e.g., dosage amount, frequency, etc.). Modifications to dosage amount may be manually set via dosage control knob 213.

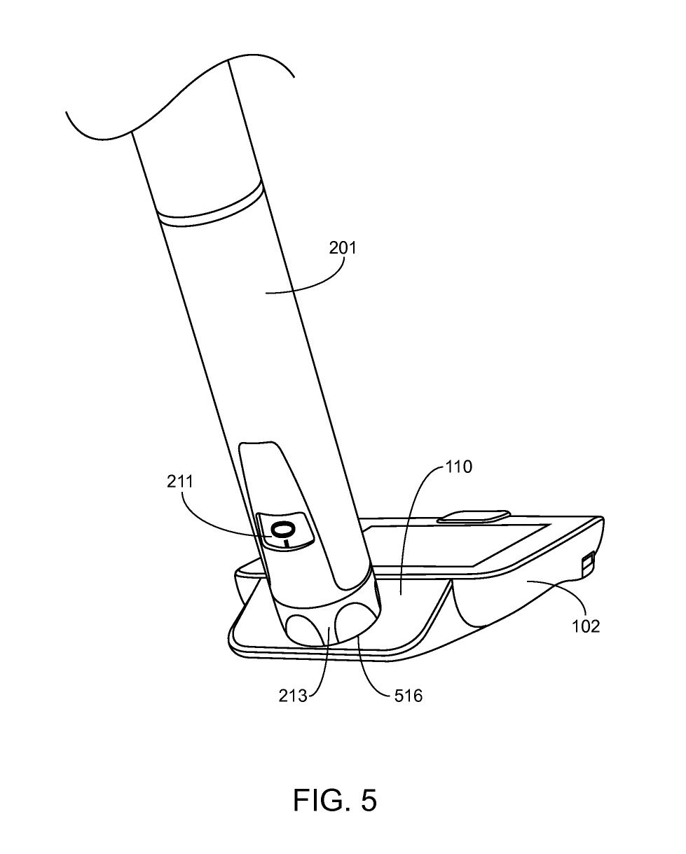

FIG. 5 illustrates the medication delivery device of FIG. 2 in direct engagement with the analyte measurement system of FIGS. 1A and 1B, in accordance with another embodiment presented herein. As shown in FIG. 5, a mechanical connection can be made between medication delivery device 201 and analyte meter 102. Such mechanical connection may be provided at interlock interface (or coupling surface) 516. In one embodiment, knob 213 and interlock interface 516 are provided with one or more matching magnets to facilitate the mechanical connection between medication delivery device 201 and analyte meter 102.

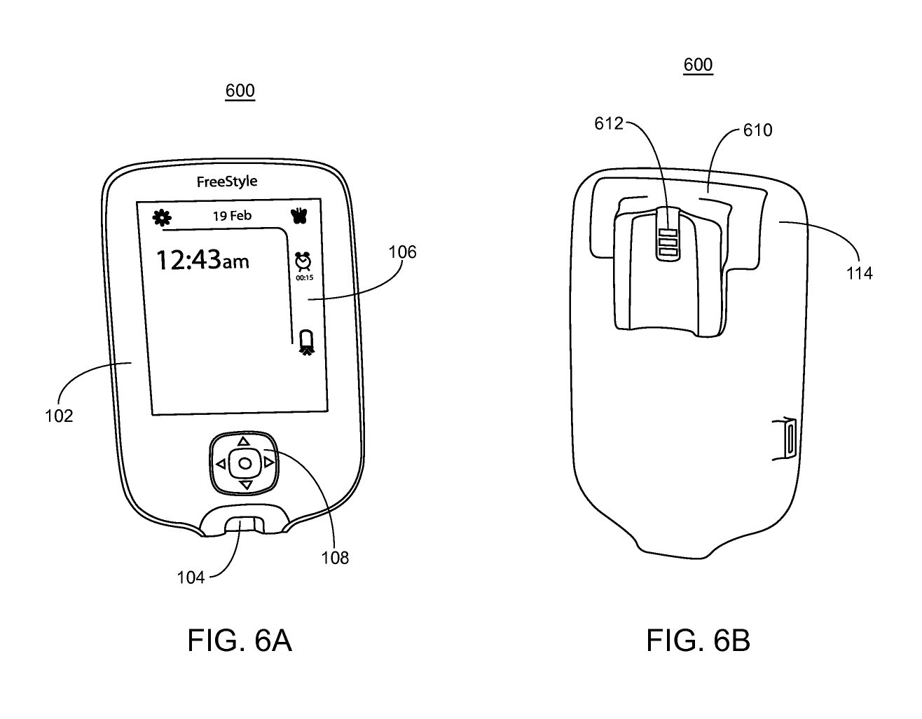

FIG. 6A provides a front-side view of an analyte measurement system 600, in accordance with another embodiment presented herein. FIG. 6B illustrates a back-side view of analyte measurement system 600. In one embodiment, analyte measurement system 600 includes an analyte meter 102 (as described above), having a test strip port 104, a display unit 106, and at least one control button 108.

In the embodiment shown, analyte measurement system 600 further includes a modular attachment 610. Modular attachment 610 is set within, and preferably interlocked within, receptacle 114 in the housing of analyte meter 102. In one embodiment, for example, a snap-fit engagement may be provided between modular attachment 610 and receptacle 114. Electrical connections (not shown) are also provided between modular attachment 610 and meter 102 in order to transmit data and/or instructions between meter 102 and modular attachment 610.

Modular attachment 610 includes a data transmission port 612, which is used to communicate with external device; such as, for example, a medication (drug) deliver device. In one embodiment, data transmission port 612 is an electrical input interface that directly couples to an electrical output interface of a medication deliver device. The terms “input” or “output,” as used herein should not be construed to limit the electrical interface to only performing “input” or “output” functions. In other embodiments, data transmission port 612 may input data from the medication deliver device and output instructions to the medication deliver device.

In alternative embodiments, the modular attachment and meter housing can take on various form factors with various attachment means. Examples of meters incorporating modular attachments are provided in U.S. Pat. No. 7,041,468, which is incorporated herein by reference in its entirety.

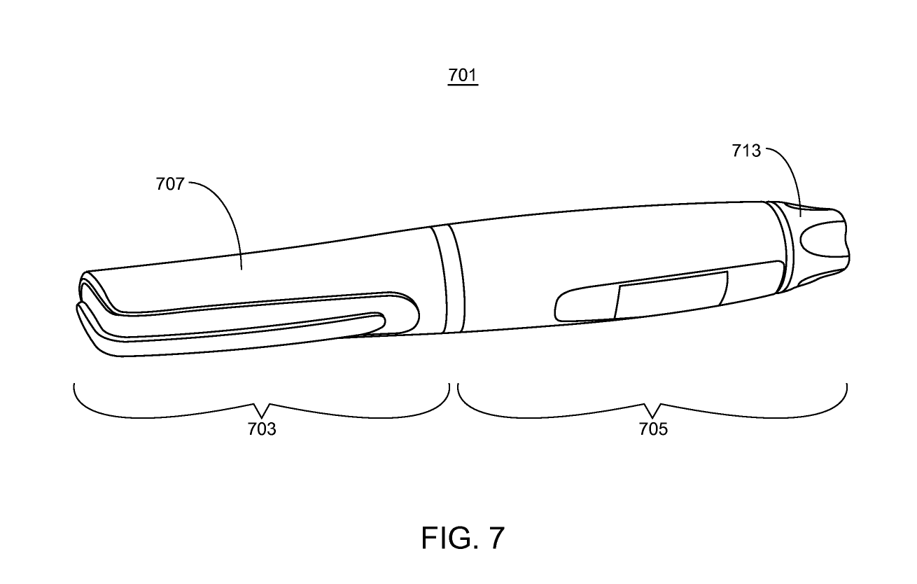

FIG. 7 illustrates a perspective view of a medication delivery device 701, in accordance with another embodiment presented herein. Medication delivery device 701 may be similar to commercially available insulin injection pens, and generally includes an injection end 703 and a data transmission end 705. Injection end 703 is shown with a removable cap 707. Underneath cap 707, the injection end 703 of medication delivery device 701 provides an injection needle or syringe. The data transmission end 705 of medication delivery device 701 includes a data transmission port 709 for communicating with data transmission port 612 on modular attachment 610. Medication delivery device 701 also includes a dosage control knob 213. Exemplary pens are described in, for example, U.S. Pat. Nos. 6,004,297; 6,235,004; 6,582,404; 7,195,616; 7,291,132; and 7,678,084; the disclosures of which are herein incorporated by reference in their entirety.

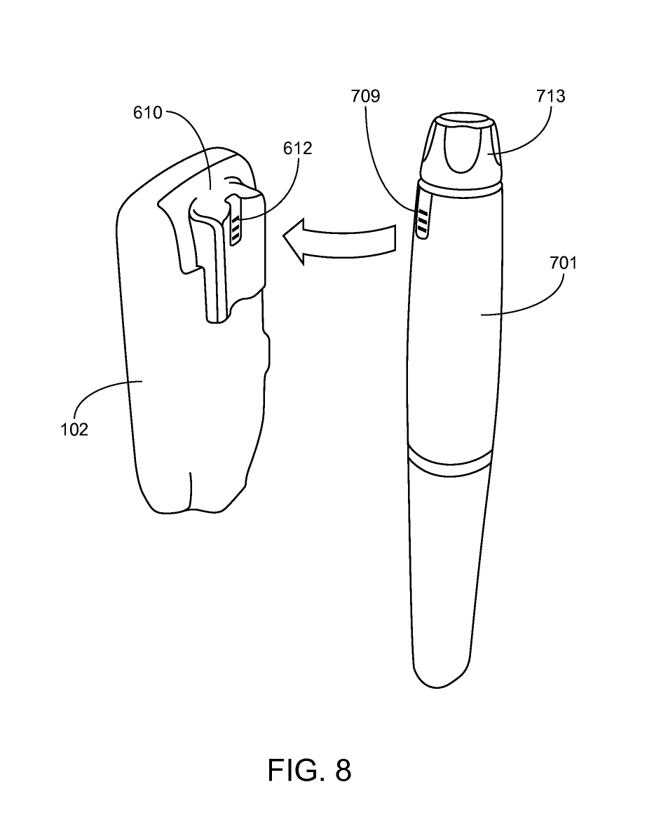

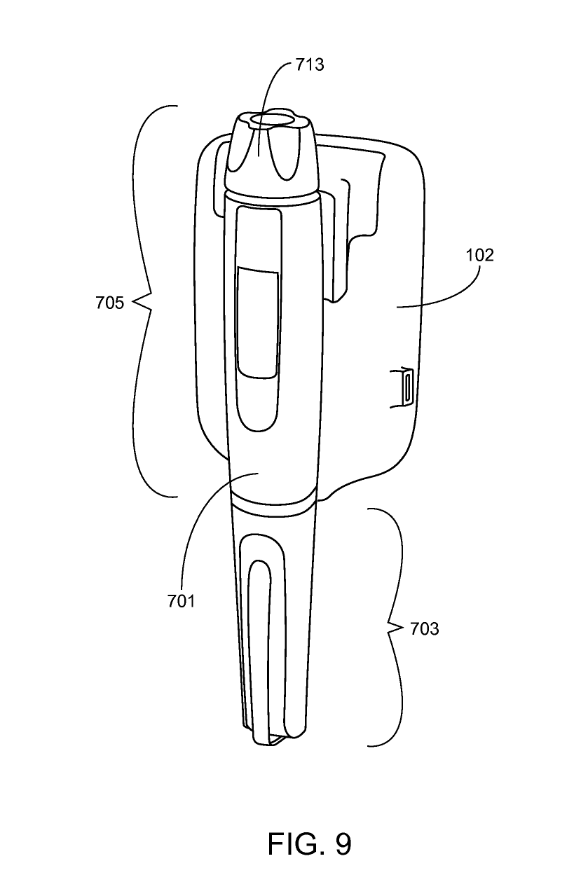

FIGS. 8 and 9 illustrates one embodiment presented herein. FIG. 9 illustrates medication delivery device 701 in direct engagement with analyte measurement system 600. In practice, medication delivery device 701 is presented to, and mechanically connected to, modular attachment 610. Such mechanical connection may be provided with one or more matching magnets to facilitate the mechanical connection between a coupling surface on the medication delivery device 701 and a coupling surface on the modular attachment 610. Data and/or instructions may then be transmitted via data transmission ports 709 and 612.

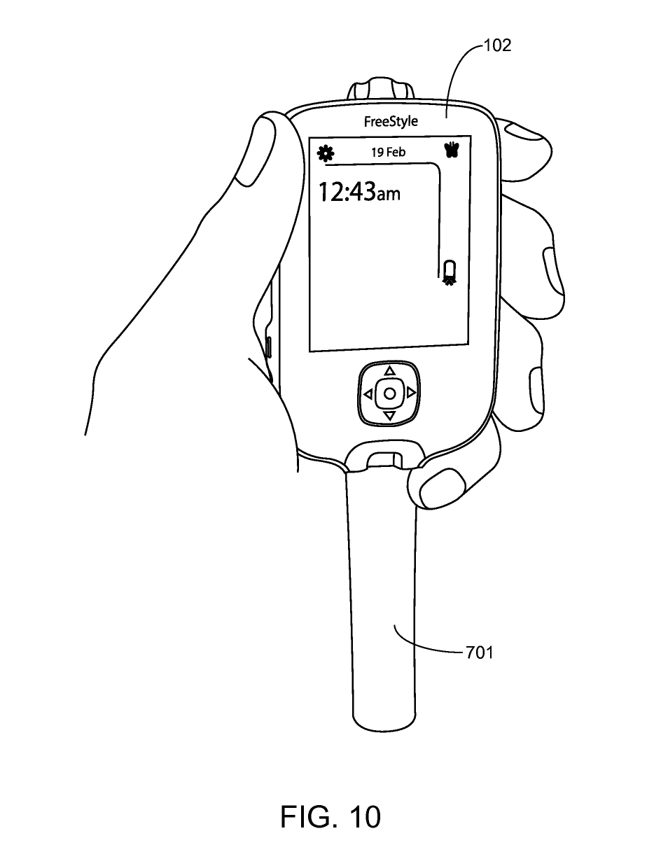

FIG. 10 illustrates a user practicing one embodiment presented herein. For example, the display screen 106 may provide the user with a means for visually reviewing dosage information from medication delivery device 701.

Integration with Medication Delivery Devices and/or Systems

In some embodiments, the analyte measurement systems disclosed herein may be included in and/or integrated with, a medication delivery device and/or system, e.g., an insulin pump module, such as an insulin pump or controller module thereof. In some embodiments the analyte measurement system is physically integrated into a medication delivery device. In other embodiments, an analyte measurement system as described herein may be configured to communicate with a medication delivery device or another component of a medication delivery system. Additional information regarding medication delivery devices and/or systems, such as, for example, integrated systems, is provided in U.S. Patent Application Publication No. US2006/0224141, published on Oct. 5, 2006, entitled “Method and System for Providing Integrated Medication Infusion and Analyte Monitoring System”, and U.S. Patent Application Publication No. US2004/0254434, published on Dec. 16, 2004, entitled “Glucose Measuring Module and Insulin Pump Combination,” the disclosure of each of which is incorporated by reference herein in its entirety. Medication delivery devices which may be provided with analyte measurement system as described herein include, e.g., a needle, syringe, pump, catheter, inhaler, transdermal patch, or combination thereof. In some embodiments, the medication delivery device or system may be in the form of a drug delivery injection pen such as a pen-type injection device incorporated within the housing of an analyte measurement system. Additional information is provided in U.S. Pat. Nos. 5,536,249 and 5,925,021, the disclosures of each of which are incorporated by reference herein in their entirety.

The embodiments presented herein provide further advantages such as: the ability to upgrade strip port modules as new test strip technologies evolve; the ability to clean or sterilize a strip port module; and the ability to allow users to replace strip port modules without returning the entire measurement system to the manufacture.

Certain embodiments relate to in vivo (e.g., continuous monitoring) systems. A continuous monitoring system typically includes a sensor that is worn or placed below the skin, a transmitter that collects glucose information from the sensor, and a receiver that collects the information from the transmitter. The sensor can collect glucose level information continuously, periodically, or at other intervals. Advantageously, a user is relieved from having to repeatedly lance his or her body to collect a blood sample once the sensor is inserted, although the sensor (e.g., an electrochemical sensor that is inserted into a body) can be replaced. U.S. Pat. No. 6,175,752, which is hereby incorporated by reference in its entirety, discloses additional examples of a continuous monitoring system.

Embodiments of the invention relate to components of a continuous monitoring system that may be replaceable. In one embodiment, the interface between the sensor and the transmitter may become contaminated. The transmitter or sensor control unit, for example, may have an interface with the sensor that has been molded to form a barrier between the transmitter's contacts and circuitry internal to the transmitter. This allows the transmitter's contacts to be washed without damaging the transmitter's circuitry. Alternatively, the contacts may be included in a replaceable port that can be replaced as needed. Similarly, the interface on the sensor may be molded to form a barrier to contamination or be replaceable.

Embodiments of the invention further extend to kits. Examples of a kit include a measurement device with one or more strip connectors. In some kits, different strip connectors or ports for different types of strips may be included. This allows the measurement device to be used with different strip form factors. The kits may also include a plurality of test strips. In certain examples, the measurement device may be configured for use with disposable test strips as well as with test strips that are configured for continuous monitoring systems. Thus, the measurement device may include a receiver to receive information from a transmitter that collects glucose information from an inserted sensor. The measurement device may also include a strip connector, such as those disclosed herein, for use with single use test strips.

### Analyte Test Strips

Analyte test strips for use with the present devices can be of any kind, size, or shape known to those skilled in the art; for example, FREESTYLE® and FREESTYLE LITE™ test strips, as well as PRECISION™ test strips sold by ABBOTT DIABETES CARE Inc. In addition to the embodiments specifically disclosed herein, the devices of the present disclosure can be configured to work with a wide variety of analyte test strips, e.g., those disclosed in U.S. patent application Ser. No. 11/461,725, filed Aug. 1, 2006; U.S. Patent Application Publication No. 2007/0095661; U.S. Patent Application Publication No. 2006/0091006; U.S. Patent Application Publication No. 2006/0025662; U.S. Patent Application Publication No. 2008/0267823; U.S. Patent Application Publication No. 2007/0108048; U.S. Patent Application Publication No. 2008/0102441; U.S. Patent Application Publication No. 2008/0066305; U.S. Patent Application Publication No. 2007/0199818; U.S. Patent Application Publication No. 2008/0148873; U.S. Patent Application Publication No. 2007/0068807; U.S. patent application Ser. No. 12/102,374, filed Apr. 14, 2008, and U.S. Patent Application Publication No. 2009/0095625; U.S. Pat. No. 6,616,819; U.S. Pat. No. 6,143,164; U.S. Pat. No. 6,592,745; U.S. Pat. No. 6,071,391 and U.S. Pat. No. 6,893,545; the disclosures of each of which are incorporated by reference herein in their entirety.

Integrated with Lancing Device

In another embodiment, an analyte measurement system may include an integrated analyte test meter and lancing device for providing a bodily fluid sample, such as a blood sample, and measuring an analyte concentration, such as a blood glucose concentration. Examples of such integrated devices include systems and devices described in US Published Application Nos. US2007/0149897 and US2008/0167578, the disclosures of each of which are incorporated herein by reference in their entirety.

### Calculation of Medication Dosage

In one embodiment, the analyte measurement system may be configured to measure the blood glucose concentration of a patient and include instructions for a long-acting insulin dosage calculation function. Periodic injection or administration of long-acting insulin may be used to maintain a baseline blood glucose concentration in a patient with Type-1 or Type-2 diabetes. In one aspect, the long-acting medication dosage calculation function may include an algorithm or routine based on the current blood glucose concentration of a diabetic patient, to compare the current measured blood glucose concentration value to a predetermined threshold or an individually tailored threshold as determined by a doctor or other treating professional to determine the appropriate dosage level for maintaining the baseline glucose level. In one embodiment, the long-acting insulin dosage calculation function may be based upon LANTUS® insulin, available from Sanofi-Aventis, also known as insulin glargine. LANTUS® is a long-acting insulin that has up to a 24 hour duration of action. Further information on LANTUS® insulin is available at the website located by placing “www” immediately in front of “.lantus.com”. Other types of long-acting insulin include Levemir® insulin available from NovoNordisk (further information is available at the website located by placing “www” immediately in front of “.levemir-us.com”. Examples of such embodiments are described in in US Published Patent Application No. US2010/01981142, the disclosure of which is incorporated herein by reference in its entirety.

### Docking Station

In another embodiment, the analyte measurement system may include a corresponding docking station or one or more other peripheral devices. The docking station may include, among others, a transmitter whereby when the analyte measurement system is docked to the docking station, the analyte measurement system and docking station may communicate over a data network with, for example, a healthcare provider, for the transfer of data or receipt of instructions or new dosage regimens. The docking station transmitter may be configured for transmission protocols including, but not limited to, cellular telephone transmission, such as code division multiple access (CDMA) or Global System for Mobile communications (GSM), internet communication, facsimile communications, and/or telephone communication. In another aspect, the docking station may also be configured to provide power for recharging a rechargeable battery of the analyte measurement system. In another aspect, the docking station may be configured for communication with a personal computer for additional storage, programming, and/or communication.

In another embodiment, a docking station such as described in U.S. Pat. No. 7,077,328 may be employed. As stated above, U.S. Pat. No. 7,077,328 is incorporated herein by reference in its entirety.

### Strip Port Configured to Receive Test Strips for Different Analytes

In another embodiment, there is provided an analyte measurement system for multichemistry testing. The test strips are for chemical analysis of a sample, and are adapted for use in combination with a measuring device having a test port and capable of performing a multiplicity of testing functionalities. Each type of test strip corresponds to at least one of the testing functionalities, and at least some types of test strips have indicators of the testing functionality on them. The test port is adapted for use in combination with a multiplicity of different types of test strips and includes a sensor capable of specifically interacting with the indicator(s) on the test strips, thereby selecting at least one of the multiplicity of testing functionalities corresponding to the type of test strip. Such system would include a strip port that can be used to read a test strip for glucose and a test strip for ketone bodies. Examples of such embodiment are provided in U.S. Pat. No. 6,773,671, which is incorporated herein by reference in it entirety.

Strip Port Configured to Receive Test Strips Having Different Dimensions and/or Electrode Configurations

In some embodiments, an analyte measurement system as described herein includes a strip port configured to receive test strips having different dimensions and/or electrode configurations, e.g., as described in the U.S. patent application Ser. No. 12/695,947 filed on Jan. 28, 2010, and entitled “Universal Test Strip Port”, the disclosure of which is incorporated by reference herein in its entirety.

### Test Strip Ejector

In some embodiments, an analyte measurement system as described herein is configured to include an optional analyte test strip ejector configured to eject an analyte test strip from a test strip port of the analyte measurement system. An analyte test strip ejector may be useful, for example, where it is desirable to eject an analyte test strip containing a sample of bodily fluid, e.g., blood, following an analyte measurement conducted using the analyte measurement system. This allows a user of the analyte measurement system to dispose of the contaminated analyte test strip without touching the analyte test strip.

In some embodiments, the analyte test strip ejector slidably engages a portion of the housing of the analyte measurement system. The analyte test strip ejector may be configured such that upon insertion of an analyte test strip into the test strip port, the analyte test strip ejector is moved rearward with respect to the test strip port and in the direction of insertion. In order to eject the analyte test strip, a user physically moves the analyte test strip ejector forward with respect to the test strip port and in the opposite of the direction of insertion. This movement in-turn exerts force upon the analyte test strip expelling it from the test strip port. Alternatively, the analyte test strip ejector may be configured such that insertion of the analyte test strip into a strip port of the analyte measurement system positions the analyte test strip ejector in a “cocked” position, e.g., by engaging a spring mechanism. The analyte measurement system may include a button, switch, or other suitable mechanism for releasing the cocked ejector from the cocked position such that it ejects the analyte test strip from the strip port of the analyte measurement system. Additional information regarding analyte test strip ejectors is provided in the U.S. patent application Ser. No. 12/695,947, filed on Jan. 28, 2010, and entitled “Universal Test Strip Port.”

### Splash-Proof Test Strip Port

In some embodiments, an analyte measurement system as described herein is configured to include a contamination resistant test strip port and/or a splash-proof test strip port. In one such embodiment, the test strip port includes one or more sealing members positioned so as to limit and/or prevent internal contamination of the test strip port with fluids and/or particles present in the environment outside the test strip port. In another embodiment, the test strip port includes an internal beveled face which can limit and/or preventingress of one or more external contaminants into the internal area of the test strip port.

Additional disclosure and examples of contamination resistant test strip ports are provided in U.S. patent application Ser. No. 12/539,217, filed Aug. 11, 2009, and entitled “Analyte Sensor Ports,” the disclosure of which is incorporated by reference herein in its entirety.

In some embodiments, the test strip ports described herein can be configured to work with (e.g., engage with or operate in connection with) additional mechanisms and/or devices designed to limit and/or prevent contamination of the internal areas of the test strip ports themselves or the internal areas of the analyte measurement system into which the test strip ports can be integrated. For example, mechanisms, devices and methods of protecting test strip port openings are described in U.S. Patent Application Publication No. US2008/0234559, and U.S. Patent Application Publication No. US2008/0119709, the disclosure of each of which is incorporated by reference herein in their entirety. Test strip ports according to the present disclosure can also be configured to be replaceable and/or disposable, and/or configured so as to limit and/or prevent contamination of the analyte measurement system in which the test strip port is integrated. Additional description is provided, for example, in U.S. Application Publication No. 2010/0064800, the disclosure of which is incorporated by reference herein it its entirety.

### Implanted Analyte Sensor

In some embodiments, an analyte measurement system as described herein may include an implanted or partially implanted analyte sensor, e.g., a system including an implanted or partially implanted glucose sensor (e.g., a continuous glucose sensor). A system including an implanted or partially implanted glucose sensor may include an analyte measurement system as described herein, which is configured to receive analyte data from the implanted or partially implanted glucose sensor either directly or through an intermediate device, e.g., an RF-powered measurement circuit coupled to an implanted or partially implanted analyte sensor. In some embodiments, where an analyte measurement system according to the present disclosure is integrated with an implanted sensor, the analyte measurement system does not include a strip port for receiving an analyte test strip. In one embodiment, the analyte measurement system may be used to calibrate the analyte monitoring system, e.g., using one point calibration or other calibration protocol. For additional information, see U.S. Pat. No. 6,175,752, the disclosure of which is incorporated by reference herein in its entirety. In some embodiments, the analyte measurement system may be configured to communicate with the implanted or partially implanted analyte sensor via Radio Frequency Identification (RFID) and provide for intermittent or periodic interrogation of the implanted analyte sensor.

Exemplary analyte monitoring systems that may be utilized in connection with the disclosed analyte measurement system include those described in U.S. Pat. No. 7,041,468; U.S. Pat. No. 5,356,786; U.S. Pat. No. 6,175,752; U.S. Pat. No. 6,560,471; U.S. Pat. No. 5,262,035; U.S. Pat. No. 6,881,551; U.S. Pat. No. 6,121,009; U.S. Pat. No. 7,167,818; U.S. Pat. No. 6,270,455; U.S. Pat. No. 6,161,095; U.S. Pat. No. 5,918,603; U.S. Pat. No. 6,144,837; U.S. Pat. No. 5,601,435; U.S. Pat. No. 5,822,715; U.S. Pat. No. 5,899,855; U.S. Pat. No. 6,071,391; U.S. Pat. No. 6,120,676; U.S. Pat. No. 6,143,164; U.S. Pat. No. 6,299,757; U.S. Pat. No. 6,338,790; U.S. Pat. No. 6,377,894; U.S. Pat. No. 6,600,997; U.S. Pat. No. 6,773,671; U.S. Pat. No. 6,514,460; U.S. Pat. No. 6,592,745; U.S. Pat. No. 5,628,890; U.S. Pat. No. 5,820,551; U.S. Pat. No. 6,736,957; U.S. Pat. No. 4,545,382; U.S. Pat. No. 4,711,245; U.S. Pat. No. 5,509,410; U.S. Pat. No. 6,540,891; U.S. Pat. No. 6,730,200; U.S. Pat. No. 6,764,581; U.S. Pat. No. 6,299,757; U.S. Pat. No. 6,461,496; U.S. Pat. No. 6,503,381; U.S. Pat. No. 6,591,125; U.S. Pat. No. 6,616,819; U.S. Pat. No. 6,618,934; U.S. Pat. No. 6,676,816; U.S. Pat. No. 6,749,740; U.S. Pat. No. 6,893,545; U.S. Pat. No. 6,942,518; U.S. Pat. No. 6,514,718; U.S. Pat. No. 5,264,014; U.S. Pat. No. 5,262,305; U.S. Pat. No. 5,320,715; U.S. Pat. No. 5,593,852; U.S. Pat. No. 6,746,582; U.S. Pat. No. 6,284,478; U.S. Pat. No. 7,299,082; U.S. Patent Application No. 61/149,639, entitled “Compact On-Body Physiological Monitoring Device and Methods Thereof”, U.S. patent application Ser. No. 11/461,725, filed Aug. 1, 2006, entitled “Analyte Sensors and Methods”; U.S. patent application Ser. No. 12/495,709, filed Jun. 30, 2009, entitled “Extruded Electrode Structures and Methods of Using Same”; U.S. Patent Application Publication No. US2004/0186365; U.S. Patent Application Publication No. 2007/0095661; U.S. Patent Application Publication No. 2006/0091006; U.S. Patent Application Publication No. 2006/0025662; U.S. Patent Application Publication No. 2008/0267823; U.S. Patent Application Publication No. 2007/0108048; U.S. Patent Application Publication No. 2008/0102441; U.S. Patent Application Publication No. 2008/0066305; U.S. Patent Application Publication No. 2007/0199818; U.S. Patent Application Publication No. 2008/0148873; U.S. Patent Application Publication No. 2007/0068807; US patent Application Publication No. 2010/0198034; and U.S. provisional application No. 61/149,639 titled “Compact On-Body Physiological Monitoring Device and Methods Thereof”, the disclosures of each of which are incorporated herein by reference in their entirety.

### Communication Interface

As discussed previously herein, an analyte measurement system according to the present disclosure can be configured to include a communication interface. In some embodiments, the communication interface includes a receiver and/or transmitter for communicating with a network and/or another device, e.g., a medication delivery device and/or a patient monitoring device, e.g., a continuous glucose monitoring device. In some embodiments, the communication interface is configured for communication with a health management system, such as the CoPilot™ system available from Abbott Diabetes Care Inc., Alameda, Calif.

The communication interface can be configured for wired or wireless communication, including, but not limited to, radio frequency (RF) communication (e.g., Radio-Frequency Identification (RFID), Zigbee communication protocols, WiFi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, an RS-232 port, or any other suitable electrical connection port to allow data communication between the analyte measurement system and other external devices such as a computer terminal (for example, at a physician's office or in hospital environment), an external medical device, such as an infusion device or including an insulin delivery device, or other devices that are configured for similar complementary data communication.

In one embodiment, the communication interface is configured for infrared communication, Bluetooth® communication, or any other suitable wireless communication protocol to enable the analyte measurement system to communicate with other devices such as infusion devices, analyte monitoring devices, computer terminals and/or networks, communication enabled mobile telephones, personal digital assistants, or any other communication devices which the patient or user of the analyte measurement system may use in conjunction therewith, in managing the treatment of a health condition, such as diabetes.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or WiFi connection to the internet at a WiFi hotspot.

In one embodiment, the analyte measurement system is configured to wirelessly communicate with a server device via the communication interface, e.g., using a common standard such as 802.11 or Bluetooth® RF protocol, or an IrDA infrared protocol. The server device could be another portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touch-screen. With such an arrangement, the user can control the analyte measurement system indirectly by interacting with the user interface(s) of the server device, which in turn interacts with the analyte measurement system across a wireless link.

In some embodiments, the communication interface is configured to automatically or semi-automatically communicate data stored in the analyte measurement system, e.g., in an optional data storage unit, with a network or server device using one or more of the communication protocols and/or mechanisms described above.

### Input Unit

As discussed previously herein, an analyte measurement system according to the present disclosure can be configured to include an input unit and/or input buttons coupled to the housing of the analyte measurement system and in communication with a controller unit and/or processor. In some embodiments, the input unit includes one or more input buttons and/or keys, wherein each input button and/or key is designated for a specific task. Alternatively, or in addition, the input unit may include one or more input buttons and/or keys that can be ‘soft buttons’ or ‘soft keys’. In the case where one or more of the input buttons and/or keys are ‘soft buttons’ or ‘soft keys’, these buttons and/or keys may be used for a variety of functions. The variety of functions may be determined based on the current mode of the analyte measurement system, and may be distinguishable to a user by the use of button instructions shown on an optional display unit of the analyte measurement system. Yet another input method may be a touch-sensitive display unit, as described in greater detail below.

In addition, in some embodiments, the input unit is configured such that a user can operate the input unit to adjust time and/or date information, as well as other features or settings associated with the operation of an analyte measurement system.

### Display Unit

As discussed previously herein, in some embodiments, an analyte measurement system according to the present disclosure includes an optional display unit or a port for coupling an optional display unit to the analyte measurement system. The display unit is in communication with a control unit and/or processor and displays the analyte test strip signals and/or results determined from the analyte test strip signals including, for example, analyte concentration, rate of change of analyte concentration, and/or the exceeding of a threshold analyte concentration (indicating, for example, hypo- or hyperglycemia).

The display unit can be a dot-matrix display, e.g., a dot-matrix LCD display. In some embodiments, the display unit includes a liquid-crystal display (LCD), thin film transistor liquid crystal display (TFT-LCD), plasma display, light-emitting diode (LED) display, seven-segment display, E-ink (electronic paper) display or combination of two or more of the above. The display unit can be configured to provide, an alphanumeric display, a graphical display, a video display, an audio display, a vibratory output, or combinations thereof. The display can be a color display. In some embodiments, the display is a backlit display.

The display unit can also be configured to provide, for example, information related to a patient's current analyte concentration as well as predictive analyte concentrations, such as trending information.

In some embodiments an input unit and a display unit are integrated into a single unit, for example, the display unit can be configured as a touch sensitive display, e.g., a touch-screen display, where the user may enter information or commands via the display area using, for example, the user's finger, a stylus or any other suitable implement, and where, the touch sensitive display is configured as the user interface in an icon driven environment, for example.

In some embodiments, the display unit does not include a screen designed to display results visually. Instead, in some embodiments the optional display unit is configured to communicate results audibly to a user of the analyte measurement system, e.g., via an integrated speaker, or via separate speakers through a headphone jack or Bluetooth® headset.

### Expanding Menu Item for Improved Readability

In some embodiments, the display unit includes a graphical user interface including a plurality of menu items, wherein the display unit is configured to provide clarification with respect to the meaning of a menu item based on a user's response speed with respect to a user input for the menu item. The menu item could take any of a variety of forms, e.g., text, icon, object or combination thereof.

In one embodiment, the graphical user interface includes a menu which in turn includes a plurality of selectable menu items. As a user navigates through the menu, e.g., by highlighting or scrolling through individual menu items, a menu item that is either unreadable or incomprehensible to the user could cause the user to pause over a menu item to be selected. In one embodiment, a choice can be presented to the user, e.g., using a dedicated physical button on an input unit, or a soft key on the menu, that offers further explanation of the item to be selected without actually selecting the item. For example, the graphical user interface can be configured such that after a pre-determined period of time a soft key offers an explanation of the menu item to be selected, e.g., by displaying a soft key with the word “MORE”, “ADDITIONAL INFORMATION”, “EXPAND”, “MAGNIFY”, “HELP” or a variation thereof displayed thereon.

The pre-determined period of time may be based on a fixed factory preset value, a value set by the user or a health care provider, or through an adaptive mechanism based on an analysis of the user's speed of navigation from past interactions with the graphical user interface. In one embodiment, the pre-determined period of time is from about 5 to about 20 seconds, e.g., from about 10 to about 15 seconds.

If the offer for clarification and/or additional information is selected, e.g., by pressing the softkey, then the menu item to be selected can be displayed in a “high emphasis” mode, e.g., where the item is displayed as if a magnifying lens is held on top of the selected item. In some embodiments, additional emphasis of the menu item to be selected can be provided, e.g., by making the menu item change color, blink, or increase in size to a pre-determined maximum limit.

### Support for On-Demand Analyte Determination Using an Analyte Sensor

In some embodiments, an analyte measurement system according to the present disclosure is further configured to receive analyte concentration data and/or signals indicative of an analyte concentration from an analyte sensor, e.g., an implanted or partially implanted analyte sensor or a radio-frequency (RF)-powered measurement circuit coupled to an implanted or partially implanted analyte sensor. In some embodiments, the analyte sensor is a self-powered analyte sensor. An analyte measurement system according to the present disclosure may include software configured to analyze signals received from the analyte sensor. Additional information related to self-powered analyte sensors and methods of communicating therewith are provided in U.S. Patent Application Publication No. 2010/0213057, the disclosure of which is incorporated by reference herein in its entirety.

### Integrated Bar Code

In an embodiment, an analyte measurement system according to the present disclosure is integrated with a barcoding system. The barcoding system may be laser or LED based, and may be used for identification of analyte test strips, patient, health care professional, etc. For example, the analyte measurement system may include a barcode reader disposed in the housing. The housing would further require a internal circuitry and a barcode scan engine for processing of a scan. Additional examples of such a bar coding system is provided in U.S. Pat. No. 7,077,328, which has been incorporated herein by reference in its entirety.

### Anti-Microbial Thin Film Cover

In an embodiment, an analyte measurement system according to the present disclosure is provided with an anti-microbial thin film cover. A common problem with many analyte measurement systems is that the housing cracks, degrades, and generally wears down due to the harsh chemicals that are used to disinfect the analyte measurement system in hospital and clinical environments. By placing an anti-microbial plastic film over the analyte measurement system, the life-cycle of the system can be prolonged because the plastic film is subjected to the disinfectants, rather than the system housing itself. When the plastic film begins to degrade, it can be removed and replaced. The plastic film also adds an additional layer of sterility to the system. The plastic film may be transparent, and applied over the display and/or user interface. One side of the plastic film would contain anti-microbial chemistry, while the back side of the plastic film would contain a thin layer of adhesive.

### Analytes

A variety of analytes can be detected and quantified using the disclosed analyte measurement system. Analytes that may be determined include, for example, acetyl choline, amylase, bilirubin, cholesterol, chorionic gonadotropin, creatine kinase (e.g., CK-MB), creatine, DNA, fructosamine, glucose, glutamine, growth hormones, hormones, ketones (e.g., ketone bodies), lactate, oxygen, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be determined. Assays suitable for determining the concentration of DNA and/or RNA are disclosed in U.S. Pat. No. 6,281,006 and U.S. Pat. No. 6,638,716, the disclosures of each of which are incorporated by reference herein in their entirety.

### CONCLUSION

The foregoing description of the invention has been presented for purposes of illustration and description. It is not intended to be exhaustive or to limit the invention to the precise form disclosed. Other modifications and variations may be possible in light of the above teachings. The embodiments were chosen and described in order to best explain the principles of the invention and its practical application, and to thereby enable others skilled in the art to best utilize the invention in various embodiments and various modifications as are suited to the particular use contemplated. It is intended that the appended claims be construed to include other alternative embodiments of the invention; including equivalent structures, components, methods, and means.

It is to be appreciated that the Detailed Description section, and not the Summary and Abstract sections, is intended to be used to interpret the claims. The Summary and Abstract sections may set forth one or more, but not all exemplary embodiments of the present invention as contemplated by the inventor(s), and thus, are not intended to limit the present invention and the appended claims in any way.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

In the description of the invention herein, it will be understood that a word appearing in the singular encompasses its plural counterpart, and a word appearing in the plural encompasses its singular counterpart, unless implicitly or explicitly understood or stated otherwise. Merely by way of example, reference to “an” or “the” “analyte” encompasses a single analyte, as well as a combination and/or mixture of two or more different analytes, reference to “a” or “the” “concentration value” encompasses a single concentration value, as well as two or more concentration values, and the like, unless implicitly or explicitly understood or stated otherwise. Further, it will be understood that for any given component described herein, any of the possible candidates or alternatives listed for that component, may generally be used individually or in combination with one another, unless implicitly or explicitly understood or stated otherwise. Additionally, it will be understood that any list of such candidates or alternatives, is merely illustrative, not limiting, unless implicitly or explicitly understood or stated otherwise.

Various terms are described to facilitate an understanding of the invention. It will be understood that a corresponding description of these various terms applies to corresponding linguistic or grammatical variations or forms of these various terms. It will also be understood that the invention is not limited to the terminology used herein, or the descriptions thereof, for the description of particular embodiments. Merely by way of example, the invention is not limited to particular analytes, bodily or tissue fluids, blood or capillary blood, or sensor constructs or usages, unless implicitly or explicitly understood or stated otherwise, as such may vary.

The publications discussed herein are provided solely for their disclosure prior to the filing date of the application. Nothing herein is to be construed as an admission that the embodiments of the invention are not entitled to antedate such publication by virtue of prior invention. Further, the dates of publication provided may be different from the actual publication dates which may need to be independently confirmed.

The detailed description of the figures refers to the accompanying drawings that illustrate an exemplary embodiment of an analyte measurement system. Other embodiments are possible. Modifications may be made to the embodiment described herein without departing from the spirit and scope of the present invention. Therefore, the following detailed description is not meant to be limiting.

Certain embodiments presented herein relate to electrical interfaces in measurement devices. Measurement devices often have electrical interfaces that allow them to electrically connect with another device or apparatus and perform an analysis of an analyte. A device that measures blood glucose levels, for example, includes electrical interfaces that allow the device to measure the blood glucose level from a small blood sample.

1. An analyte measurement system, comprising:
an analyte meter having a meter housing and a receptacle formed in the meter housing;
a modular attachment set within the receptacle formed in the meter housing, wherein the modular attachment includes an optical reader; and
a medication delivery device, wherein the medication delivery device includes a dosage display module;
wherein the optical reader is configured to read the dosage display module on the medication delivery device.

2. The analyte measurement system of claim 1, wherein the dosage display module provides a human-readable output.

3. The analyte measurement system of claim 1, wherein the dosage display module provide a machine-readable output.

4. The analyte measurement system of claim 3, wherein the machine-readable output is a barcode.

5. The analyte measurement system of claim 3, wherein the machine-readable output is a two-dimensional barcode.

6. The analyte measurement system of claim 1, wherein dosage display module is an infrared transmitter, and the optical reader is an infrared optical reader.

7. The analyte measurement system of claim 1, wherein the modular attachment includes a coupling surface to interface with the medication delivery device.

8. The analyte measurement system of claim 7, wherein the modular attachment and the medication delivery device are removably attached via the coupling surface.

9. The analyte measurement system of claim 7, wherein the coupling surface includes at least one magnet.

10. The analyte measurement system of claim 1, wherein the dosage display module is an LED display.

11. The analyte measurement system of claim 1, wherein the analyte meter includes a display module to display dosage information received from the medication delivery device.

12. The analyte measurement system of claim 1, wherein the analyte meter includes a software module which identifies the medication delivery device.

13. The analyte measurement system of claim 1, wherein the analyte meter includes a software module which provides dosage instructions to the medication delivery device.

14. An analyte measurement system, comprising:
an analyte meter having a meter housing and a receptacle formed in the meter housing;
a modular attachment set within the receptacle formed in the meter housing, wherein the modular attachment includes an electrical input interface and a coupling surface; and
a medication delivery device, wherein the medication delivery device includes an electrical output interface and a coupling surface;
wherein the coupling surface on the medication delivery device is configured to removably attach to the coupling surface on the modular attachment; and
wherein the electrical output interface on the medication delivery device is configured to provide dosage data to the electrical input interface on the modular attachment.

15. The analyte measurement system of claim 14, wherein each coupling surface includes at least one magnet.

16. The analyte measurement system of claim 14, wherein the analyte meter includes a display module to display dosage information received from the medication delivery device.

17. The analyte measurement system of claim 14, wherein the analyte meter includes a software module which identifies the medication delivery device.

18. The analyte measurement system of claim 14, wherein the analyte meter includes a software module which provides dosage instructions to the medication delivery device.

19. A modular attachment for an analyte meter in an analyte measurement system, comprising:
a housing configured to fit within a receptacle formed in the analyte meter; and
an optical reader configured to read a dosage display module on a medication delivery device.

20. The modular attachment of claim 19, wherein the optical reader is configured to read a human-readable output from the dosage display module on the medication delivery device.

21. The modular attachment of claim 19, wherein the optical reader is configured to read a machine-readable output from the dosage display module on the medication delivery device.

22. The modular attachment of claim 21, wherein the machine-readable output is a barcode.

23. The modular attachment of claim 21, wherein the machine-readable output is a two-dimensional barcode.

24. The modular attachment of claim 19, wherein the optical reader is an infrared optical reader.

25. The modular attachment of claim 19, further comprising a coupling surface to interface with the medication delivery device.

26. The modular attachment of claim 25, wherein the modular attachment and the medication delivery device are removably attached via the coupling surface.

27. The modular attachment of claim 25, wherein the coupling surface includes at least one magnet.

28. The modular attachment of claim 19, wherein the dosage display module is an LED display.
